(19) United States
(12) Patent Application Publication
(10) Pub. No.: US 2024/0254144 A1
(43) Pub. Date: Aug. 1, 2024

(54) SALT FORM OF PYRROLOTRIAZINE COMPOUND, CRYSTAL FORM THEREOF, AND PREPARATION METHOD THEREFOR

(71) Applicant: JUMBO DRUG BANK CO., LTD., Chengdu, Sichuan (CN)

(72) Inventors: Lingyun WU, Shanghai (CN); Xiawei WEI, Chengdu, Sichuan (CN); Xu YOU, Shanghai (CN); Xiongbin XU, Shanghai (CN); Ning JIANG, Chengdu, Sichuan (CN); Shuhui CHEN, Shanghai (CN)

(21) Appl. No.: 18/558,731

(22) PCT Filed: May 7, 2022
(86) PCT No.: PCT/CN2022/091452

§ 371 Date: Nov. 2, 2023

(30) Foreign Application Priority Data
May 8, 2021 (CN) 202110501179.9

(51) Int. Cl. C07D 519/00; A61K 31/53; A61P 35/00

(52) U.S. Cl. CPC C07D 519/00; A61K 31/53; A61P 35/00

(57) ABSTRACT

A pyrrolotriazine compound, e.g., the compound of formula (II), a salt form thereof, a crystal form thereof, a preparation method therefor, and an application thereof are provided. An application in preparation of MNK1/2 inhibitor drugs and/or in preparation of drugs for treating colorectal cancer are also provided.

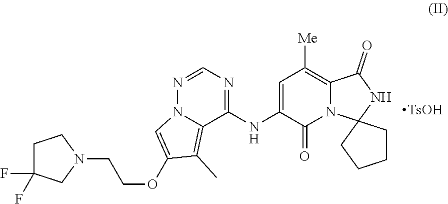

### PRIORITY AND RELATED APPLICATIONS

The present disclosure claims priority to the prior application with the patent application No. 202110501179.9 and entitled “SALT FORM OF PYRROLOTRIAZINE COMPOUND, CRYSTAL FORM THEREOF, AND PREPARATION METHOD THEREFOR” filed to China National Intellectual Property Administration on May 8, 2021, which is incorporated herein by reference in its entirety.

### TECHNICAL FIELD

The present disclosure relates to a salt form of a pyrrolotriazine compound, a crystal form thereof, a preparation method therefor, and use thereof, and in particular to a compound of formula (II) and a crystal form thereof.

### BACKGROUND

Mitogen-activated protein kinase interacting kinase (MAP kinase interacting kinase, or MNK for short) is a serine/threonine protein kinase. There are four subtypes for human MNK, namely MNK1a and MNK1b expressed by MNK1 gene, and MNK2a and MNK2b expressed by MNK2 gene, respectively. All the 4 subtypes contain a nuclear localization signal (NLS) sequence and a sequence for binding to eIF4G at the N-terminal, and are thus capable of entering into a cell nucleus to play a role, and recognizing and binding to downstream eIF4E. The subtypes MNK1a and MNK2a have a binding site for MAPK at the C-terminal, and can be activated by upstream ERK and p38 phosphorylation. The nuclear export signal (NES) sequence at C-terminal of MNK1a makes it widely present in the cytoplasm, whereas the other 3 subtypes are mostly present in the nucleus. Eukaryotic initiation factor 4E (eIF4E) is a cap-binding protein, and it can specifically recognize the cap structure at the 5′ end of mRNA and is an important initiation factor in protein translation. S209-phosphorylated eIF4E can promote the translation of downstream proteins, mainly including c-MYC, cyclin D1, VEGF, FGF, and anti-apoptosis proteins such as mcl-1 and Bcl-2. The expression of eIF4E is up-regulated in lung cancer, colorectal cancer, gastric cancer, pancreatic duct carcinoma and other malignant tumors. MNK is the only kinase known to be able to phosphorylate eIF4E. In addition, MNK is located at an intersection of multiple signaling pathways involved in tumors and immune, such as RAS and T cell receptor (TCR), and can selectively control transcription of regulators of anti-tumor immune responses. MNK activity and activation of eIF4E are critical for tumor development and progression, but are not necessarily for normal cells. Therefore, a selective MNK inhibitor is expected to become an anti-tumor drug with low toxicity. EFT508 (WO2015/200481; WO2016/172010; WO2017/075394; WO2017/075412; WO2017/087808; WO2017/117052; WO2018/152117; WO2018/218038) is a selective oral MNK inhibitor being developed by EFFECTOR THERAPEUTICS INC. Research has shown that the eFT508 can selectively inhibit the expression of PD-1, LAG3 and IL-10, and improve the function of cytotoxic T cells without affecting the proliferation of normal T cells. Pre-clinical studies have found that the combination of eFT508 and PD-1 monoclonal antibody can enhance efficacy and increase response rate. The phase I clinical trial of the drug has been completed, and it showed good safety; currently, the monotherapy for hematological tumors and trending prostate cancer is in phase II clinical trial, the combined therapy with avelumab monoclonal antibody for microsatellite stable colorectal cancer (MSS CRC) is in phase II clinical trial, and the combined therapy with PD-1/PD-L1 therapy (for patients who are treated with PD-1/PD-L1 therapy alone before and have had disease progression or no complete or partial alleviation) for solid tumors is in phase II clinical trial.

### SUMMARY

The present disclosure provides a compound of formula (II),

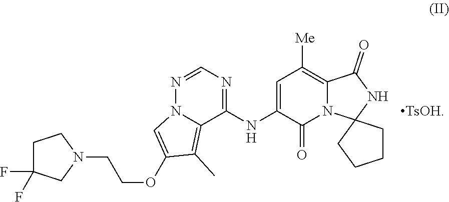

The present disclosure further provides crystal form A of the compound of formula (II), wherein an X-ray powder diffraction pattern of the crystal form A has characteristic diffraction peaks at the following 2θ angles: 7.78±0.20°, 11.38±0.20°, and 20.58±0.20°;

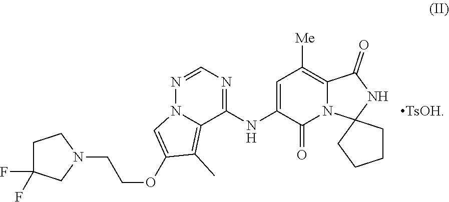

In some embodiments of the present disclosure, the X-ray powder diffraction pattern of the crystal form A described above has characteristic diffraction peaks at the following 2θ angles: 7.78±0.20°, 9.44±0.20°, 11.38±0.20°, 19.84±0.20°, 20.58±0.20°, 21.56±0.20°, 22.86±0.20°, and 24.82±0.20°.

In some embodiments of the present disclosure, the X-ray powder diffraction pattern of the crystal form A described above has characteristic diffraction peaks at the following 2θ angles: 6.58±0.20°, 7.78±0.20°, 9.44±0.20°, 11.38±0.20°, 14.38±0.20°, 18.66±0.20°, 19.84±0.20°, 20.58±0.20°, 21.56±0.20°, 22.86±0.20°, 23.54±0.20°, and 24.82±0.20°.

In some embodiments of the present disclosure, the X-ray powder diffraction pattern of the crystal form A described above has characteristic diffraction peaks at the following 2θ angles: 4.78°±0.20°, 6.58°±0.20°, 7.78°±0.20°, 9.44°±0.20°, 11.38°±0.20°, 13.48°±0.20°, 14.38°±0.20°, 14.800±0.200, 16.42°±0.20°, 17.00°±0.20°, 17.32°±0.20°, 18.34°±0.20°, 18.66°±0.20°, 19.08°±0.20°, 19.60°±0.20°, 19.840±0.200, 20.280±0.200, 20.580±0.200, 21.560±0.200, 21.840±0.200, 22.520±0.200, 22.860±0.200, 23.26°±0.20°, 23.54°±0.20°, 24.46°±0.20°, 24.82°±0.20°, 25.50°±0.20°, 26.04°±0.20°, 26.58°±0.20°, 27.42°±0.20°, 27.82°±0.20°, 28.07°±0.20°, 28.42°±0.20°, 29.08°±0.20°, 29.66°±0.20°, 30.08°±0.20°, 31.20°±0.20°, 31.42°±0.20°, 38.22°±0.20°, and 39.04°±0.20°.

In some embodiments of the present disclosure, the X-ray powder diffraction pattern of the crystal form A described above has characteristic diffraction peaks at the following 2θ angles: 4.78°±0.10°, 6.58°±0.10°, 7.78°±0.10°, 9.44°±0.10°, 11.38°±0.10°, 13.48°±0.10°, 14.38°±0.10°, 14.80°±0.10°, 16.42°±0.10°, 17.00°±0.10°, 17.32°±0.10°, 18.34°±0.10°, 18.66°±0.10°, 19.08°±0.10°, 19.60°±0.10°, 19.84°±0.10°, 20.28°±0.10°, 20.58°±0.10°, 21.56°±0.10°, 21.84°±0.10°, 22.52°±0.10°, 22.86°±0.10°, 23.26°±0.10°, 23.54°±0.10°, 24.46°±0.10°, 24.82°±0.10°, 25.50°±0.10°, 26.04°±0.10°, 26.58°±0.10°, 27.42°±0.10°, 27.82°±0.10°, 28.07°±0.10°, 28.42°±0.10°, 29.08°±0.10°, 29.66°±0.10°, 30.08°±0.10°, 31.20°±0.10°, 31.42°±0.10°, 38.22°±0.100, and 39.04°±0.10°.

In some embodiments of the present disclosure, the X-ray powder diffraction pattern of the crystal form A described above has characteristic diffraction peaks at the following 2θ angles: 4.78°, 6.58°, 7.78°, 9.44 11.38°, 13.48°, 14.38°, 14.80°, 16.42°, 17.00°, 17.32°, 18.34°, 18.66°, 19.08°, 19.60°, 19.84°, 20.28°, 20.58°, 21.56°, 21.84°, 22.52°, 22.86°, 23.26°, 23.54°, 24.46°, 24.82°, 25.50°, 26.04°, 26.58°, 27.42°, 27.82°, 28.07°, 28.42°, 29.08°, 29.66°, 30.08°, 31.20°, 31.42°, 38.22°, and 39.04°.

In some embodiments of the present disclosure, the X-ray powder diffraction pattern of the crystal form A described above has characteristic diffraction peaks at the following 2θ angles: 4.78°±0.20°, and/or 6.58°±0.20°, and/or 7.78°±0.20°, and/or 9.44°±0.20°, and/or 11.38°±0.20°, and/or 13.48°±0.20°, and/or 14.38°±0.20°, and/or 14.80°±0.20°, and/or 16.42°±0.20°, and/or 17.00°±0.20°, and/or 17.32°±0.20°, and/or 18.34°±0.20°, and/or 18.66°±0.20°, and/or 19.08°±0.20°, and/or 19.60°±0.20°, and/or 19.84°±0.20°, and/or 20.28°±0.20°, and/or 20.58°±0.20°, and/or 21.56°±0.20°, and/or 21.84°±0.20°, and/or 22.52°±0.20°, and/or 22.86°±0.20°, and/or 23.26°±0.20°, and/or 23.54°±0.20°, and/or 24.46°±0.20°, and/or 24.82°±0.20°, and/or 25.50°±0.20°, and/or 26.04°±0.20°, and/or 26.58° 0.20°, and/or 27.42° 0.20°, and/or 27.82° 0.20°, and/or 28.07° 0.20°, and/or 28.42° 0.20°, and/or 29.08°±0.20°, and/or 29.66°±0.20°, and/or 30.08°±0.20°, and/or 31.20°±0.20°, and/or 31.42°±0.20°, and/or 38.22°±0.20°, and/or 39.04°±0.20°.

In some embodiments of the present disclosure, the X-ray powder diffraction pattern of the crystal form A described above has characteristic diffraction peaks at the following 2θ angles: 4.78°±0.10°, and/or 6.58°±0.10°, and/or 7.78°±0.10°, and/or 9.44°±0.10°, and/or 11.38°±0.10°, and/or 13.48°±0.10°, and/or 14.38°±0.10°, and/or 14.80°±0.10°, and/or 16.42°±0.10°, and/or 17.00°±0.10°, and/or 17.32°±0.10°, and/or 18.34°±0.10°, and/or 18.66°±0.10°, and/or 19.08°±0.10°, and/or 19.60°±0.10°, and/or 19.84°±0.10°, and/or 20.28°±0.10°, and/or 20.58°±0.10°, and/or 21.56°±0.10°, and/or 21.84°±0.10°, and/or 22.52°±0.10°, and/or 22.86°±0.10°, and/or 23.26°±0.10°, and/or 23.54°±0.10°, and/or 24.46°±0.10°, and/or 24.82°±0.10°, and/or 25.50°±0.10°, and/or 26.04°±0.10°, and/or 26.58°±0.10°, and/or 27.42°±0.10°, and/or 27.82°±0.10°, and/or 28.07°±0.10°, and/or 28.42°±0.10°, and/or 29.08°±0.10°, and/or 29.66°±0.10°, and/or 30.08°±0.10°, and/or 31.20°±0.10°, and/or 31.42°±0.10°, and/or 38.22°±0.10°, and/or 39.04°±0.10°.

In some embodiments of the present disclosure, the XRPD pattern of the crystal form A described above is substantially as shown in FIG. 1.

In some embodiments of the present disclosure, the XRPD of the crystal form A described above is determined using Cu-Kα radiation.

In some embodiments of the present disclosure, XRPD pattern analysis data for the crystal form A described above are shown in Table 1.

TABLE 1
XRPD analysis data for crystal form
A of compound of formula (II)

| | | | Inter- | |
|---|---|---|---|---|
| | 2θ | Peak | planar | Relative |
| | angle | intensity | spacing | intensity |
| No. | (°) | (cts) | (Å) | (%) |
| 1 | 4.78 | 124 | 18.46 | 9.8 |
| 2 | 6.58 | 104 | 13.42 | 8.2 |
| 3 | 7.78 | 357 | 11.35 | 28.2 |
| 4 | 9.44 | 245 | 9.36 | 19.3 |
| 5 | 11.38 | 1265 | 7.77 | 100.0 |
| 6 | 13.48 | 93 | 6.57 | 7.3 |
| 7 | 14.38 | 310 | 6.15 | 24.5 |
| 8 | 14.80 | 251 | 5.98 | 19.8 |
| 9 | 16.42 | 137 | 5.39 | 10.9 |
| 10 | 17.00 | 144 | 5.21 | 11.4 |
| 11 | 17.32 | 91 | 5.12 | 7.2 |
| 12 | 18.34 | 291 | 4.83 | 23.0 |
| 13 | 18.66 | 349 | 4.75 | 27.6 |
| 14 | 19.08 | 136 | 4.65 | 10.8 |
| 15 | 19.60 | 269 | 4.53 | 21.2 |
| 16 | 19.84 | 514 | 4.47 | 40.6 |
| 17 | 20.28 | 167 | 4.38 | 13.2 |
| 18 | 20.58 | 726 | 4.31 | 57.4 |
| 19 | 21.56 | 667 | 4.12 | 52.7 |
| 20 | 21.84 | 341 | 4.07 | 26.9 |
| 21 | 22.52 | 240 | 3.94 | 19.0 |
| 22 | 22.86 | 640 | 3.89 | 50.6 |
| 23 | 23.26 | 301 | 3.82 | 23.8 |
| 24 | 23.54 | 426 | 3.78 | 33.6 |
| 25 | 24.46 | 68 | 3.64 | 5.4 |
| 26 | 24.82 | 453 | 3.58 | 35.8 |
| 27 | 25.50 | 93 | 3.49 | 7.4 |
| 28 | 26.04 | 179 | 3.42 | 14.1 |
| 29 | 26.58 | 214 | 3.35 | 16.9 |
| 30 | 27.42 | 97 | 3.25 | 7.7 |
| 31 | 27.82 | 190 | 3.20 | 15.0 |
| 32 | 28.07 | 85 | 3.18 | 6.7 |
| 33 | 28.42 | 81 | 3.14 | 6.4 |
| 34 | 29.08 | 91 | 3.07 | 7.2 |
| 35 | 29.66 | 81 | 3.01 | 6.4 |
| 36 | 30.08 | 90 | 2.97 | 7.1 |
| 37 | 31.20 | 71 | 2.86 | 5.6 |
| 38 | 31.42 | 63 | 2.84 | 5.0 |
| 39 | 38.22 | 36 | 2.35 | 2.8 |
| 40 | 39.04 | 57 | 2.31 | 4.5 |

In some embodiments of the present disclosure, a differential scanning calorimetry curve of the crystal form A described above has an endothermic peak at 287.17±3° C.

In some embodiments of the present disclosure, a DSC profile of the crystal form A described above is shown in FIG. 2.

In some embodiments of the present disclosure, a thermogravimetric analysis curve (TGA) of the crystal form A described above shows a weight loss of 0.07500 at 200.0±3° C.

In some embodiments of the present disclosure, a TGA profile of the crystal form A described above is shown in FIG. 3.

The present disclosure further provides a preparation method for the crystal form A of the compound of formula (II), comprising the following steps:
(a) adding the compound of formula (II) to a solvent to form a suspension;
(b) stirring the suspension at 40-55° C. for 2-25 h; and
(c) filtering the suspension and then drying the filter cake in vacuum at 30-45° C. for 10-24 h; wherein the solvent is selected from methanol, acetonitrile, and tert-butyl methyl ether.

The present disclosure provides crystal form B of the compound of formula (II), wherein an X-ray powder diffraction (XRPD) pattern of the crystal form B has characteristic diffraction peaks at the following 2θ angles: 7.55±0.20°, 15.13±0.20°, and 19.82±0.20°;

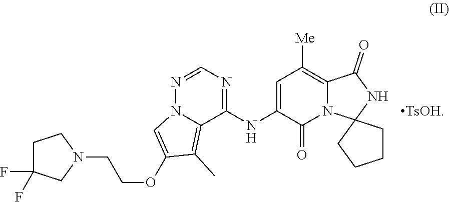

In some embodiments of the present disclosure, the X-ray powder diffraction pattern of the crystal form B described above has characteristic diffraction peaks at the following 2θ angles: 6.89±0.20°, 7.55±0.20°, 9.50±0.20°, 11.35±0.20°, 12.72±0.20°, 15.13±0.20°, 19.82±0.20°, and 26.63±0.20°.

In some embodiments of the present disclosure, the X-ray powder diffraction pattern of the crystal form B described above has characteristic diffraction peaks at the following 2θ angles: 6.89±0.20°, 7.55±0.20°, 9.50±0.20°, 11.35±0.20°, 12.24±0.20°, 12.72±0.20°, 15.13±0.20°, 18.94±0.20°, 19.82±0.20°, 23.25±0.20°, 26.63±0.20°, and 27.27±0.20°.

In some embodiments of the present disclosure, the X-ray powder diffraction pattern of the crystal form B described above has characteristic diffraction peaks at the following 2θ angles: 4.98°+0.20°, 6.89°±0.20°7.55°±0.20°8.46°±0.20°9.50°±0.20°10.12°±0.20°11.35°±0.20°12.24°±0.20°12.72°±0.20°, 14.05°±0.20°, 15.13°±0.20°, 15.65°±0.20°, 16.20°±0.20°, 17.79°±0.20°, 18.94°±0.20° °19.82°±0.20°20.76°±0.20°21.61°±0.20°23.25°±0.20°23.87°±0.20°26.09°±0.20°26.63°±0.20°27.27°±0.20°28.45°±0.20°29.12°±0.20°30.95°±0.20°32.32°±0.20°34.62°±0.200, and 38.41°±0.20°.

In some embodiments of the present disclosure, the X-ray powder diffraction pattern of the crystal form B described above has characteristic diffraction peaks at the following 2θ angles: 4.98°+0.10°, 6.89°±0.10°7.55°±0.10°8.46°±0.10°9.50°±0.10°10.12°±0.10°11.35°±0.10°12.24°±0.10°12.72°±0.10°, 14.05°±0.10°, 15.13°±0.10°, 15.65°±0.10°, 16.20°±0.10°, 17.79°±0.10°, 18.94°±0.10°, 19.82°±0.10°20.76°±0.10°21.61°±0.10°23.25°±0.10°23.87°±0.10°26.09°±0.10°26.63°±0.10°27.27°±0.10°, 28.45°±0.10°, 29.12°±0.10°, 30.95°±0.10°, 32.32°±0.10°, 34.62°±0.10°, and 38.41°±0.10°.

In some embodiments of the present disclosure, the X-ray powder diffraction pattern of the crystal form B described above has characteristic diffraction peaks at the following 2θ angles: 4.98°, 6.89°, 7.55°, 8.46°, 9.50°, 10.12°, 11.35°, 12.24°, 12.72°, 14.05°, 15.13°, 15.65°, 16.20°, 17.79°, 18.94°, 19.82°, 20.76°, 21.61°, 23.25°, 23.87°, 26.09°, 26.63°, 27.27°, 28.45°, 29.12°, 30.95°, 32.32°, 34.62°, and 38.41°.

In some embodiments of the present disclosure, the X-ray powder diffraction pattern of the crystal form B described above has characteristic diffraction peaks at the following 2θ angles: 4.98°±0.20°, and/or 6.89°±0.20°, and/or 7.55°±0.20°, and/or 8.46°±0.20°, and/or 9.50°±0.20°, and/or 10.12°±0.20°, and/or 11.35°±0.20°, and/or 12.24°±0.20°, and/or 12.72°±0.20°, and/or 14.05°±0.20°, and/or 15.13°±0.20°, and/or 15.65°±0.20°, and/or 16.20°±0.20°, and/or 17.79°±0.20°, and/or 18.94°±0.20°, and/or 19.82°±0.20°, and/or 20.76°±0.20°, and/or 21.61°±0.20°, and/or 23.25°±0.20°, and/or 23.87°±0.20°, and/or 26.09°±0.20°, and/or 26.63°±0.20°, and/or 27.27°±0.20°, and/or 28.45°±0.20°, and/or 29.12°±0.20°, and/or 30.95°±0.20°, and/or 32.32°±0.20°, and/or 34.62°±0.20°, and/or 38.41° 0.20°.

In some embodiments of the present disclosure, the X-ray powder diffraction pattern of the crystal form B described above has characteristic diffraction peaks at the following 2θ angles: 4.98°±0.10°, and/or 6.89°±0.10°, and/or 7.55°±0.10°, and/or 8.46°±0.10°, and/or 9.50°±0.10°, and/or 10.12°±0.10°, and/or 11.35°±0.10°, and/or 12.24°±0.10°, and/or 12.72°±0.10°, and/or 14.05°±0.10°, and/or 15.13°±0.10°, and/or 15.65°±0.10°, and/or 16.20°±0.10°, and/or 17.79°±0.10°, and/or 18.94°±0.10°, and/or 19.82°±0.10°, and/or 20.76°±0.10°, and/or 21.61°±0.10°, and/or 23.25°±0.10°, and/or 23.87°±0.10°, and/or 26.09°±0.10°, and/or 26.63°±0.10°, and/or 27.27°±0.10°, and/or 28.45°±0.10°, and/or 29.12°±0.10°, and/or 30.95°±0.10°, and/or 32.32°±0.10°, and/or 34.62°±0.10°, and/or 38.41°±0.10°.

In some embodiments of the present disclosure, the XRPD pattern of the crystal form B described above is substantially as shown in FIG. 4.

In some embodiments of the present disclosure, the XRPD of the crystal form B described above is determined using Cu-Kα radiation.

In some embodiments of the present disclosure, XRPD pattern analysis data for the crystal form B described above are shown in Table 2.

TABLE 2
XRPD analysis data for crystal form
B of compound of formula (II)

| | | | Inter- | |
|---|---|---|---|---|
| | 2θ | Peak | planar | Relative |
| | angle | intensity | spacing | intensity |
| No. | (°) | (cts) | (Å) | (%) |
| 1 | 4.98 | 72.32 | 17.74 | 2.97 |
| 2 | 6.89 | 896.22 | 12.82 | 36.80 |
| 3 | 7.55 | 1530.49 | 11.71 | 62.85 |
| 4 | 8.46 | 157.42 | 10.46 | 6.46 |
| 5 | 9.50 | 859.72 | 9.31 | 35.30 |
| 6 | 10.12 | 135.06 | 8.74 | 5.55 |
| 7 | 11.35 | 889.45 | 7.80 | 36.52 |
| 8 | 12.24 | 510.42 | 7.23 | 20.96 |
| 9 | 12.72 | 666.73 | 6.96 | 27.38 |
| 10 | 14.05 | 259.16 | 6.31 | 10.64 |
| 11 | 15.13 | 2435.25 | 5.86 | 100.00 |
| 12 | 15.65 | 242.34 | 5.66 | 9.95 |
| 13 | 16.20 | 272.35 | 5.47 | 11.18 |
| 14 | 17.79 | 95.51 | 4.99 | 3.92 |
| 15 | 18.94 | 579.19 | 4.69 | 23.78 |
| 16 | 19.82 | 1532.59 | 4.48 | 62.93 |
| 17 | 20.76 | 150.45 | 4.28 | 6.18 |
| 18 | 21.61 | 244.84 | 4.11 | 10.05 |
| 19 | 23.25 | 347.03 | 3.83 | 14.25 |
| 20 | 23.87 | 274.20 | 3.73 | 11.26 |
| 21 | 26.09 | 263.02 | 3.42 | 10.80 |
| 22 | 26.63 | 913.50 | 3.35 | 37.51 |
| 23 | 27.27 | 351.00 | 3.27 | 14.41 |
| 24 | 28.45 | 138.33 | 3.14 | 5.68 |
| 25 | 29.12 | 70.84 | 3.07 | 2.91 |
| 26 | 30.95 | 54.09 | 2.89 | 2.22 |
| 27 | 32.32 | 32.02 | 2.77 | 1.31 |
| 28 | 34.62 | 16.98 | 2.59 | 0.70 |
| 29 | 38.41 | 68.55 | 2.34 | 2.81 |

In some embodiments of the present disclosure, a differential scanning calorimetry curve of the crystal form B described above has an endothermic peak at 300.0±3° C.

In some embodiments of the present disclosure, a DSC profile of the crystal form B described above is shown in FIG. 5.

The present disclosure further provides a preparation method for the crystal form B of the compound of formula (II), comprising the following steps:
(a) adding the compound of formula (II) to a solvent to form a suspension;
(b) stirring the suspension at 40-55° C. for 2-25 h; and
(c) filtering the suspension and then drying the filter cake in vacuum at 30-45° C. for 10-24 h; wherein the solvent is selected from ethanol and n-heptane.

The present disclosure further provides use of the compound of formula (II) described above, the crystal form A described above and the crystal form B described above or crystal forms prepared by the method described above for manufacturing an MNK1/2 inhibitor medicament.

The present disclosure further provides use of the compound of formula (II) described above, the crystal form A described above and the crystal form B described above or crystal forms prepared by the method described above for manufacturing a medicament for the treatment of colorectal cancer.

### Technical Effects

The compound of formula (I) of the present disclosure has high selectivity for MNK1/2 and also has significant inhibitory activity against the kinases; in addition, the compound has good membrane permeability and has excellent pharmacokinetic and pharmacodynamic properties. Each of crystal forms of the compound of formula (II) is stable, has little influence by light, heat and humidity, and has good in-vivo efficacy and a wide pharmaceutical prospect.

### Definitions and Description

Unless otherwise stated, the following terms and phrases used herein are intended to have the following meanings. A particular phrase or term, unless otherwise specifically defined, should not be considered as uncertain or unclear, but construed according to its common meaning. When referring to a trade name, it is intended to refer to its corresponding commercial product or its active ingredient.

The intermediate compounds of the present disclosure can be prepared by a variety of synthetic methods well known to those skilled in the art, including the specific embodiments listed below, embodiments formed by combinations thereof with other chemical synthetic methods, and equivalent substitutions thereof known to those skilled in the art. Preferred embodiments include, but are not limited to, the examples disclosed herein.

The chemical reactions of the specific embodiments of the present disclosure are carried out in a suitable solvent that must be suitable for the chemical changes in the present disclosure and the reagents and materials required. In order to acquire the compounds of the present disclosure, it is sometimes necessary for those skilled in the art to modify or select a synthesis procedure or a reaction scheme based on the existing embodiments.

The structures of the compounds of the present disclosure can be confirmed by conventional methods well known to those skilled in the art, and if the present disclosure relates to an absolute configuration of the compound, the absolute configuration can be confirmed by means of conventional techniques in the art. For example, in the single crystal X-ray diffraction (SXRD) method, intensity data of diffraction of the single crystal grown are collected with a Bruker D8 venture diffractometer, the light source is CuKα radiation, and the scanning mode is (p/Q scanning; after related data are collected, the direct method (Shelxs97) is further employed to analyze the crystal structure, and thus the absolute configuration can be confirmed.

The present disclosure is described in detail below by way of examples, which are not intended to limit the present disclosure in any way.

All solvents used in the present disclosure are commercially available and can be used without further purification.

The following abbreviations are used in the present disclosure: DCM represents dichloromethane; DMF represents N,N-dimethylformamide; DMSO represents dimethyl sulfoxide; EtOH represents ethanol; MeOH represents methanol; 2-MeTHF represents 2-methyltetrahydrofuran; ACN represents acetonitrile; toluene represents methylbenzene; EtOAc represents ethyl acetate; THF represents tetrahydrofuran; H₂O represents water; TosOH represents p-toluenesulfonic acid.

Compounds are named according to conventional nomenclature rules in the art or using ChemDraw® software, and supplier's catalog names are given for commercially available compounds.

### BRIEF DESCRIPTION OF THE DRAWINGS

FIG. 1 shows an XRPD pattern of crystal form A of a compound of formula (II);

FIG. 2 shows a DSC profile of the crystal form A of the compound of formula (II);

FIG. 3 shows a TGA profile of the crystal form A of the compound of formula (II);

FIG. 4 shows an XRPD pattern of crystal form B of the compound of formula (II);

FIG. 5 shows a DSC profile of the crystal form B of the compound of formula (II);

FIG. 6 shows a DVS profile of the crystal form A of the compound of formula (II); and

FIG. 7 shows an ellipsoid plot of a single crystal of the compound of formula (II).

### Instrument Parameters

The X-ray powder diffractometer (XRPD) method for the crystal form A of the present disclosure has test parameters shown in Table 3.

TABLE 3
XRPD test parameters for crystal form A
XRPD test parameters

| Parameter | Set value |
|---|---|
| Model | DX-2700BH |
| X-ray | Cu, k-α: 1.54184 Å; |
| X-ray light tube settings | 40 kV, 30 mA |
| Divergence slit | 1 mm |
| Primary scattering slit | 28 mm |
| Secondary slit | 28 mm |
| Detector slit | 0.3 mm |
| Anti-scatter slit | 1 mm |
| Scanning axis | θ s-θ d |
| Scanning range 2θ (°) | 3~40 |
| Scanning time (s) | 0.5 |
| Scanning step size 2θ (°) | 0.02 |

The differential scanning calorimeter (DSC) method for the crystal form A of the present disclosure has test parameters shown in Table 4.

TABLE 4
DSC test parameters for crystal form A
DSC test parameters

| Model of instrument | Mettler DSC1 |
|---|---|
| Method | Linear heating |
| Crucible type | High-pressure gold-plated crucible, |
| | closed |
| Temperature range | 40-350° C. |
| Scanning speed | 10 |
| (° C./min) | |
| Protective gas | Nitrogen |

The thermal gravimetric analyzer (TGA) method for the crystal form A of the present disclosure has test parameters shown in Table 5.

TABLE 5
TGA test parameters for crystal form A
TGA test parameters

| Model of instrument | TGA 550 |
|---|---|
| Method | Linear heating |
| Sample tray | Aluminum tray, open |
| Temperature range | 40~500° C. |
| Scanning speed (° C./min) | 10 |
| Protective gas | Nitrogen |

The dynamic vapor sorption (DVS) method for the crystal form A of the present disclosure has test parameters shown in Table 6.

TABLE 6
DVS test parameters for crystal form A

| Manufacturer and model | |
|---|---|
| of instrument | SMS/DVS intrinsic |
| Test condition | Weighing about 10 mg of |
| | sample for testing |
| Temperature | 25° C. |
| Equilibration | dm/dt: 0.01%/min |
| Drying | Drying at 25° C. and 0% RH |
| | for 2 h |
| RH (%) test gradient | 5% RH |
| Range of RH (%) test | 0%~95%~0% RH |
| gradient | |

The evaluation categories of hygroscopicity are shown in Table 7 below:

TABLE 7
Evaluation category table of hygroscopicity

| Categories of hygroscopicity | ΔW % |
|---|---|
| Deliquescence | Absorbing sufficient water |
| | to form a solution |
| Very hygroscopic | ΔW % ≥ 15% |
| Hygroscopic | 15% > ΔW % ≥ 2% |
| Slightly hygroscopic | 2% > ΔW % ≥ 0.2% |
| Non-hygroscopic or hardly | ΔW % < 0.2% |
| hygroscopic | |

Note:
ΔW % represents the hygroscopic weight gain of the test compound at 25 ± 1° C. and 80 ± 2% RH.

The X-ray powder diffractometer (XRPD) method for the crystal form B of the present disclosure has test parameters shown in Table 8.

TABLE 8
XRPD test parameters for crystal form B
XRPD test parameters

| Parameter | Set value |
|---|---|
| Model | X′Pert³ |
| X-ray | Cu, kα, Kα1 (Å): 1.54060; Kα2 (Å): 1.54443; |
| X-ray light tube settings | 45 kV, 40 mA |
| Divergence slit | Fixed ⅛° |
| Anti-scatter slit | P7.5 |
| Scanning mode | Continuous |
| Scanning range 2θ (°) | 3~40 |
| Scanning time of each step | 46.665 |
| (s) | |
| Scanning step size 2θ (°) | 0.0263 |

The differential scanning calorimeter (DSC) method for the crystal form B of the present disclosure has test parameters shown in Table 9.

TABLE 9
DSC test parameters for crystal form B
DSC test parameters

| Model of instrument | TA Discovery DSC 2500 |
|---|---|
| Method | Linear heating |
| Sample tray | Aluminum tray, closed |
| Temperature range | 25-330° C. |
| Scanning speed | 10 |
| (° C./min) | |
| Protective gas | Nitrogen |

### Example 1: Preparation of Compound of Formula (I)

### Synthetic Route:

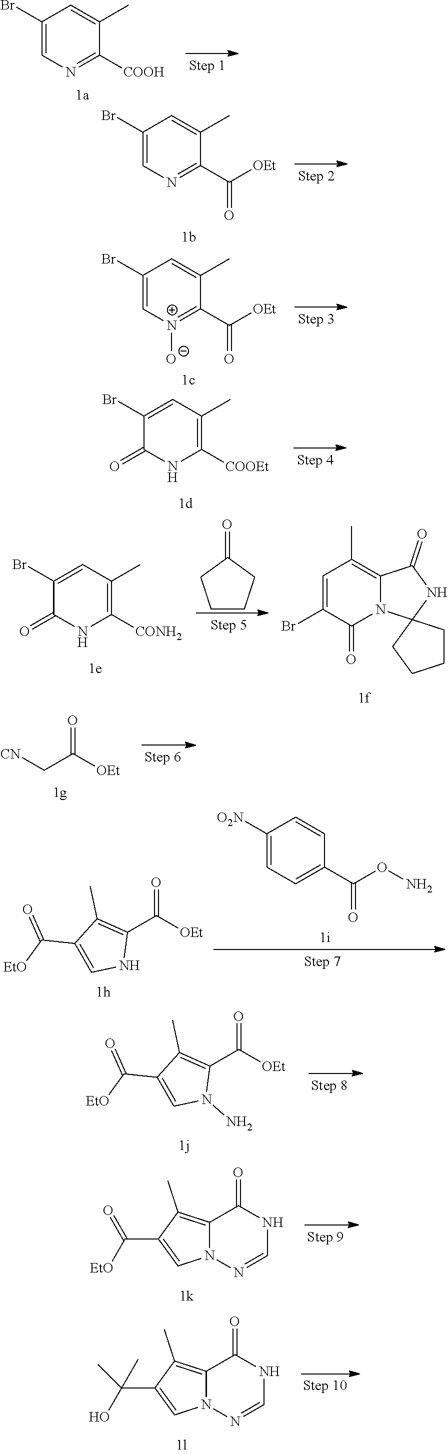

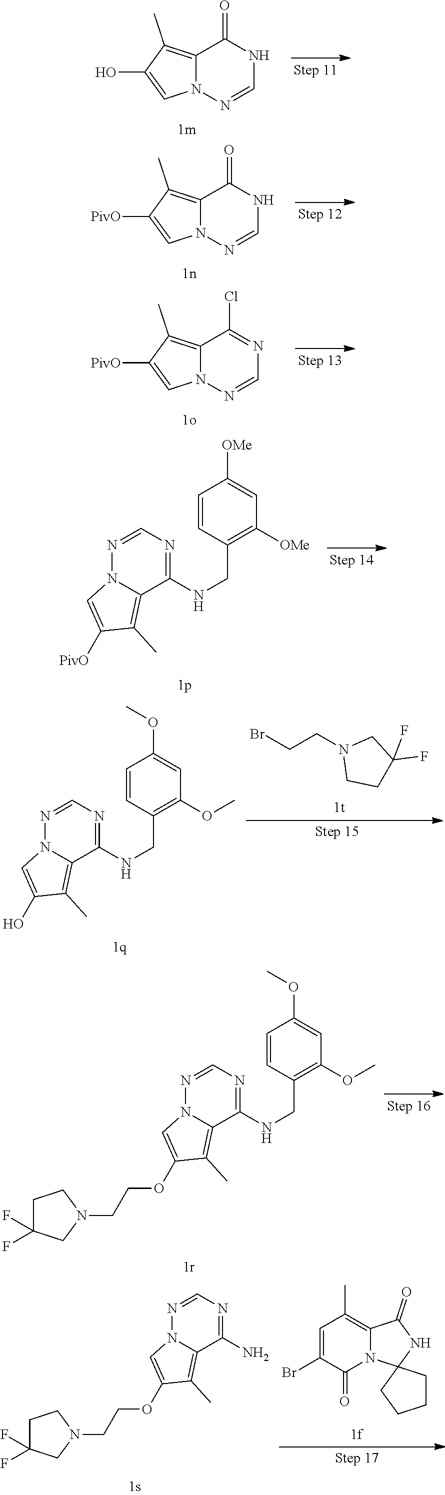

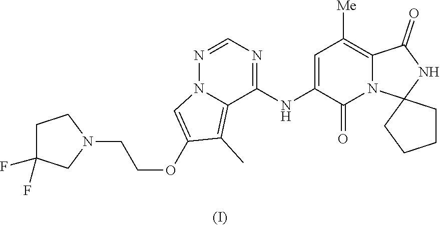 [SMILES: Cc1cc(Nc2ncnn3cc(OCCN4CCC(F)(F)C4)c(C)c23)c(=O)n2c1C(=O)NC21CCCC1 ; "(I)"]

### Step 1

Compound 1a (100 g, 462 mmol) was dissolved in ethanol (500 mL), and concentrated sulfuric acid (49.94 g, 509 mmol, purity: 98%) was added dropwise at 0° C. The reaction liquid was stirred at 95° C. for 16 h. After the reaction was completed, the reaction liquid was concentrated under reduced pressure to remove most of the ethanol. Water (300 mL) was added to the concentrate, and the mixed solution was extracted with ethyl acetate (250 mL×3). The combined organic phase was washed with saturated sodium bicarbonate (300 mL), dried over anhydrous sodium sulfate, and filtered, and the filtrate was concentrated under reduced pressure to give compound 1b. ¹H NMR (400 MHz, CDCl₃) δ 8.60 (s, 1H), 7.78 (s, 1H), 4.48-4.42 (m, 2H), 2.58 (s, 3H), 1.43 (t, J=7.2 Hz, 3H).

### Step 2

Compound 1b (10.0 g, 41.0 mmol) was dissolved in dichloromethane (200 mL), and trifluoroacetic anhydride (17.2 g, 81.9 mmol) and urea hydrogen peroxide (8.09 g, 86.0 mmol) were added under stirring at 0° C. The reaction liquid was warmed to 25° C. and stirred for 16 h. Water (200 mL) was added to the reaction liquid, and the mixed solution was extracted with dichloromethane (100 mL×3). The organic phases were combined, washed successively with saturated sodium bicarbonate (200 mL×2) and saturated brine (500 mL), dried over anhydrous sodium sulfate, filtered, and concentrated under reduced pressure to give compound 1c. ¹H NMR (400 MHz, CDCl₃) δ 8.23 (s, 1H), 7.29 (s, 1H), 4.52-4.45 (m, 2H), 2.29 (s, 3H), 1.41 (t, J=7.2 Hz, 3H).

### Step 3

Compound 1c (22.0 g, 84.6 mmol) was dissolved in N,N-dimethylformamide (130 mL), and trifluoroacetic anhydride (35.5 g, 169 mmol) was added under stirring at 0° C. The reaction liquid was stirred at 50° C. for 1 h. Water (200 mL) was added to the reaction liquid, and the mixed solution was extracted with ethyl acetate (100 mL×4). The organic phases were combined, washed successively with saturated sodium bicarbonate (200 mL×3) and saturated brine (150 mL×3), dried over anhydrous sodium sulfate, filtered, and concentrated under reduced pressure. The crude product was added to a mixed liquid of petroleum ether/ethyl acetate (8/1, 90 mL), and the mixture was stirred at room temperature for 2 h and then filtered to give compound 1d. ¹H NMR (400 MHz, CDCl₃) δ 9.89 (br s, 1H), 7.75 (s, 1H), 4.46-4.41 (m, 2H), 2.44 (s, 3H), 1.43 (t, J=7.2 Hz, 3H).

### Step 4

Compound 1d (2.00 g, 7.69 mmol) was dissolved in ethanol (20 mL), and aqueous ammonia (16.2 g, 115 mmol, purity: 25%) was added. The reaction liquid was stirred at 40° C. for 16 h. The reaction liquid was concentrated under reduced pressure, and the crude product was added to a mixed liquid of methanol/dichloromethane (1/5, 48 mL). The mixture was stirred overnight at room temperature, then filtered and washed with dichloromethane (5 mL×2). The filter cake was concentrated under reduced pressure to give compound 1e. MS-ESI, [M+H]⁺, calculated: 231 and 233, found: 231 and 233.

### Step 5

Compound 1e (500 mg, 1.97 mmol) and cyclopentanone (664 mg, 7.89 mmol) were dissolved in anhydrous dioxane (6 mL), and concentrated sulfuric acid (98.7 mg, 0.986 mmol, purity: 98%) was added dropwise to the reaction liquid. The reaction liquid was stirred at 95° C. for 3 h. The reaction liquid was concentrated under reduced pressure to remove a part of dioxane (about 3 mL), and then filtered. n-Hexane (10 mL) was added to the collected filter cake, and the mixture was stirred at room temperature for 2 h and filtered. The filter cake was dried in vacuum for 2 h to give compound 1f. MS-ESI, [M+H]⁺, calculated: 297 and 299, found: 297 and 299. ¹H NMR (400 MHz, DMSO-d₆) δ 10.16 (s, 1H), 8.02 (s, 1H), 2.71-2.78 (m, 2H), 2.37 (s, 3H), 1.91-1.93 (m, 2H), 1.79-1.84 (m, 2H), 1.63-1.67 (m, 2H).

### Step 6

Compound 1g (50 g, 0.442 mol), 1,8-diazabicyclo[5.4.0]undec-7-ene (67.3 g, 0.442 mol) were dissolved in tetrahydrofuran (500 mL). The reaction liquid was heated to 55° C., and acetaldehyde (9.74 g, 0.221 mol) was added to the reaction liquid at this temperature. The reaction liquid was stirred at 55° C. for 18 h. Then the reaction liquid was cooled to 22° C., and quenched with acetic acid (25 mL). The reaction liquid was concentrated under reduced pressure, and the residue was dissolved in ethyl acetate (1000 mL) and diluted hydrochloric acid (1000 mL, 1 M). The organic phase was retained after liquid separation, and the aqueous phase was extracted with ethyl acetate (300 mL×3). The organic phases were combined, washed successively with saturated sodium bicarbonate solution (100 mL) and brine (200 mL), dried over anhydrous sodium sulfate, filtered, and concentrated, and the crude product was separated by column chromatography (4/1, petroleum ether/ethyl acetate, Rf=0.56) to give compound 1h. MS-ESI, [M+H]⁺, calculated: 226, found: 226. ¹H NMR (400 MHz, CDCl₃) δ 9.29 (br s, 1H), 7.48 (d, J=3.2 Hz, 1H), 4.35 (q, J=7.2 Hz, 2H), 4.29 (q, J=7.2 Hz, 2H), 2.61 (s, 3H), 1.38 (t, J=7.2 Hz, 3H), 1.35 (m, J=7.2 Hz, 3H).

### Step 7

Compound 1h (11.0 g, 48.8 mmol) was dissolved in N-methylpyrrolidone (60 mL), and potassium tert-butoxide (6.03 g, 53.7 mmol) was added to the reaction liquid. After the reaction liquid was stirred at 25° C. for 0.5 h, a solution of compound 1i (9.78 g, 53.7 mmol) in N-methylpyrrolidone (30 mL) was added. The reaction liquid was further stirred for 20 h. The reaction liquid was washed with water (200 mL) and extracted with ethyl acetate (200 mL×3). The organic phases were combined, washed with saturated brine (20 mL×3), dried over anhydrous sodium sulfate, filtered, and concentrated, and the crude product was separated by column chromatography (4/1, petroleum ether/ethyl acetate, Rf=0.55) to give compound 1j. MS-ESI, [M+H]⁺, calculated: 241, found: 241. ¹H NMR (400 MHz, CDCl₃) δ 7.49 (s, 1H), 4.35 (q, J=7.2 Hz, 2H), 4.27 (q, J=7.2 Hz, 2H), 2.57 (s, 3H), 1.40 (t, J=7.2 Hz, 3H), 1.34 (t, J=7.2 Hz, 3H).

### Step 8

Compound 1j (10.2 g, 42.5 mmol) was dissolved in formamide (120 mL), and phosphoric acid (832 mg, 8.49 mmol) was added to the reaction liquid. The reaction liquid was stirred at 125° C. for 16 h. Then the reaction liquid was cooled to 22° C., and a large amount of white solid was precipitated out here. The mixture was filtered, and the collected filter cake was added to a mixed solution of petroleum ether/ethyl acetate (1/1, 100 mL). The resulting mixture was stirred at 30° C. for 0.5 h and then filtered to give compound 1k. MS-ESI, [M+H]⁺, calculated: 222, found: 222. ¹H NMR (400 MHz, DMSO-d₆) δ 7.90 (s, 1H), 7.84 (s, 1H), 4.23 (q, J=7.2 Hz, 2H), 2.61 (s, 3H), 1.28 (t, J=7.2 Hz, 3H).

### Step 9

Compound 1k (4.00 g, 18.0 mmol) was dissolved in anhydrous tetrahydrofuran (50 mL). Methylmagnesium bromide (30.1 mL, 90.3 mmol) was added dropwise to the reaction liquid at 25° C. After the dropwise addition was completed, the reaction liquid was warmed to 25° C. and stirred for 15 h. The reaction liquid was quenched with saturated ammonium chloride solution (100 mL), and extracted with ethyl acetate (50 mL×5). The organic phases were combined, washed with saturated brine (10 mL×1), dried over anhydrous sodium sulfate, filtered, and concentrated under reduced pressure, and the crude product was separated by thin layer chromatography (2/1, petroleum ether/ethyl acetate, Rf=0.39) to give compound 11.

MS-ESI, [M+H]⁺, calculated: 208, found: 208. ¹H NMR (400 MHz, CDCl₃) δ 7.30 (br s, 1H), 7.24 (br s, 1H), 2.59 (s, 3H), 1.54 (s, 6H).

### Step 10

Compound 1l (1.00 g, 4.83 mmol) and hydrogen peroxide (4.64 mL, 48.26 mmol, content: 30%) were dissolved in anhydrous tetrahydrofuran (30 mL). A cold solution of methanesulfonic acid (3.44 mL, 48.26 mmol) in water (10 mL) was added dropwise to the reaction liquid at 0° C. The reaction liquid was stirred at 0° C. for 1 h. The reaction liquid was quenched with 10% aqueous sodium sulfite solution (15 mL) until potassium iodide starch test paper showed negative. The solution was extracted with ethyl acetate (50 mL×3). The organic phases were combined, washed with saturated brine (10 mL×1), dried over anhydrous sodium sulfate, filtered, and concentrated under reduced pressure, and the crude product was separated by column chromatography (2/1, petroleum ether/ethyl acetate, Rf=0.38) to give compound 1m. MS-ESI, [M+H]⁺, calculated: 166, found: 166.

### Step 11

Compound 1m (400 mg, 2.42 mmol) was dissolved in anhydrous tetrahydrofuran (10 mL), and triethylamine (0.674 mL, 4.84 mmol) and pivaloyl chloride (350 mg, 4.84 mmol) were added to the reaction liquid. The reaction liquid was stirred at 0° C. for 1 h, washed with water (10 mL), and extracted with ethyl acetate (10 mL×5). The organic phases were combined, washed with saturated brine (10 mL×1), dried over anhydrous sodium sulfate, filtered, and concentrated under reduced pressure, and the crude product was separated by thin layer chromatography (2/1, petroleum ether/ethyl acetate, Rf=0.63) to give compound 1n. MS-ESI, [M+H]⁺, calculated: 250, found: 250. ¹H NMR (400 MHz, CD₃OD) δ 7.61 (s, 1H), 7.47 (s, 1H), 2.34 (s, 3H), 1.38 (s, 9H).

### Step 12

Compound 1n (450 mg, 1.81 mmol) was dissolved in phosphorus oxychloride (8.85 mL). The reaction liquid was stirred at 100° C. for 1 h. The reaction liquid was then cooled to room temperature and poured into saturated ammonium bicarbonate solution (300 mL). The mixture was extracted with dichloromethane (50 mL×3), and the organic phases were combined, washed with saturated brine (20 mL×1), dried over anhydrous sodium sulfate, filtered, and concentrated to give compound 1o. MS-ESI, [M+H]⁺, calculated: 268, found: 268.

### Step 13

Compound 1o (2.00 g, 7.47 mmol), 2,4-dimethoxybenzylamine (1.87 g, 11.21 mmol) and triethylamine (2.27 g, 22.4 mmol) were dissolved in anhydrous tetrahydrofuran (30 mL). The reaction liquid was stirred at 70° C. for 1 h. The reaction liquid was then concentrated under reduced pressure to give crude compound 1p. MS-ESI, [M+H]⁺, calculated: 399, found: 399.

### Step 14

Compound 1p (3.50 g, 8.78 mmol) was dissolved in methanol (3 mL) and tetrahydrofuran (20 mL), and a solution of sodium hydroxide (703 mg, 17.6 mmol) in water (20 mL) was added to the reaction liquid. The reaction liquid was stirred at 25° C. for 0.5 h. The reaction liquid was concentrated to remove the organic solvent, and the aqueous phase was adjusted to pH=7 with diluted aqueous hydrochloric acid solution (1 M) and the mixture was extracted with dichloromethane (50 mL×3). The organic phases were combined, washed with saturated brine (10 mL×1), dried over anhydrous sodium sulfate, filtered, and concentrated under reduced pressure, and the crude product was separated by column chromatography (2/1, petroleum ether/ethyl acetate, Rf=0.32) to give compound 1q. MS-ESI, [M+H]⁺, calculated: 315, found: 315. ¹H NMR (400 MHz, CD₃OD) δ 7.67 (s, 1H), 7.21 (d, J=8.4 Hz, 1H), 7.07 (s, 1H), 6.56 (d, J=2.4 Hz, 1H), 6.47 (dd, J=2.4, 8.4 Hz, 1H), 4.66 (s, 2H), 3.90 (s, 3H), 3.79 (s, 3H), 2.36 (s, 3H).

### Step 15

Compound 1t (250 mg, 795 μmol) was dissolved in N,N-dimethylformamide (4 mL), and then compound 1q (187 mg, 875 μmol) and sodium hydroxide (63.6 mg, 1.59 mmol) were added. The reaction liquid was stirred at 50° C. for 0.5 h. After the reaction was completed, water (50 mL) was added to the reaction liquid for dilution, and the mixed solution was extracted with ethyl acetate (30 mL×3). The combined organic phase was dried over anhydrous sodium sulfate and filtered, and the filtrate was concentrated under reduced pressure. The residue was separated by column chromatography (1:1, petroleum ether/ethyl acetate, Rf=0.1) to give compound 1r. MS-ESI, [M+H]⁺, calculated: 448, found: 448.

### Step 16

Compound 1r (300 mg, 670 μmol) was dissolved in trifluoroacetic acid (3.0 mL), and the reaction liquid was stirred at 100° C. for 1 h. After the reaction was completed, the reaction liquid was concentrated, and the residue was purified by high performance liquid chromatography (hydrochloric acid system) to give the hydrochloride of compound 1s. MS-ESI, [M+H]⁺, calculated: 298, found: 298.

### Step 17

The hydrochloride of compound 1s (90 mg) and compound 1f (72 mg, 241 μmol) were dissolved in anhydrous dioxane (2 mL), and then cesium carbonate (250 mg, 766 μmol) and methanesulfonate(2-dicyclohexylphosphino-3,6-dimethoxy-2,4,6-tri-isopropyl-1,1-biphenyl)(2-amino-1,1-biphenyl-2-yl)palladium(II) (20 mg, 21.9 μmol) were added. The reaction liquid was stirred at 105° C. for 12 h under nitrogen atmosphere. The reaction liquid was concentrated under reduced pressure, and the residue was separated and purified by column chromatography (10:1, dichloromethane/methanol, Rf=0.3) to give a crude compound. A mixed solution of methanol and ethanol (4/1, 10 mL) was added to the crude product, and the mixture was stirred at 20° C. for 16 h and filtered. The filter cake was washed with methanol (2 mL×2) and water (2 mL×2) and then dried to give a compound of formula (I). MS-ESI, [M+H]⁺, calculated: 514, found: 514. ¹H NMR (400 MHz, DMSO-d₆) δ 10.00 (s, 1H), 8.84 (s, 1H), 8.64 (s, 1H), 8.08 (s, 1H), 7.70 (s, 1H), 4.08 (t, J=5.6 Hz, 2H), 3.00 (t, J=13.5 Hz, 2H), 2.91-2.78 (m, 6H), 2.47 (s, 3H), 2.46 (s, 3H), 2.31-2.18 (m, 2H), 2.04-1.92 (m, 2H), 1.91-1.78 (m, 2H), 1.76-1.62 (m, 2H).

### Example 2: Preparation of Compound of Formula (II) and Single Crystal Growth Thereof

### Synthetic Route:

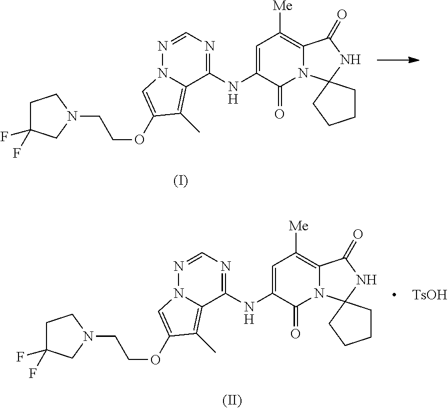

### Step 1

The compound of formula (I) (2 g, 3.89 μmol) and hexafluoroisopropanol (40 mL) were stirred and mixed well, and p-toluenesulfonic acid monohydrate (814.89 mg, 4.28 mmol) was added to the solution. The reaction liquid was stirred at 40° C. for 3 h and added dropwise to isopropanol (160 mL). The mixed solution was filtered, and the filter cake was dried in vacuum to give a compound of formula (II). ¹H NMR (400 MHz, DMSO-d₆) δ 10.02 (s, 1H), 8.86 (br s, 1H), 8.64 (s, 1H), 8.11 (s, 1H), 7.78 (s, 1H), 7.48 (d, J=8.0 Hz, 2H), 7.10 (d, J=8.0 Hz, 2H), 4.31 (br d, J=4.4 Hz, 2H), 4.08-3.62 (m, 6H), 2.89-2.78 (m, 2H), 2.72-2.57 (m, 2H), 2.51 (br s, 3H), 2.46 (s, 3H), 2.28 (s, 3H), 1.95-1.97 (m, 2H), 1.89-1.79 (m, 2H), 1.73-1.64 (m, 2H). MS-ESI, [M+H]⁺, calculated: 514, found: 514.

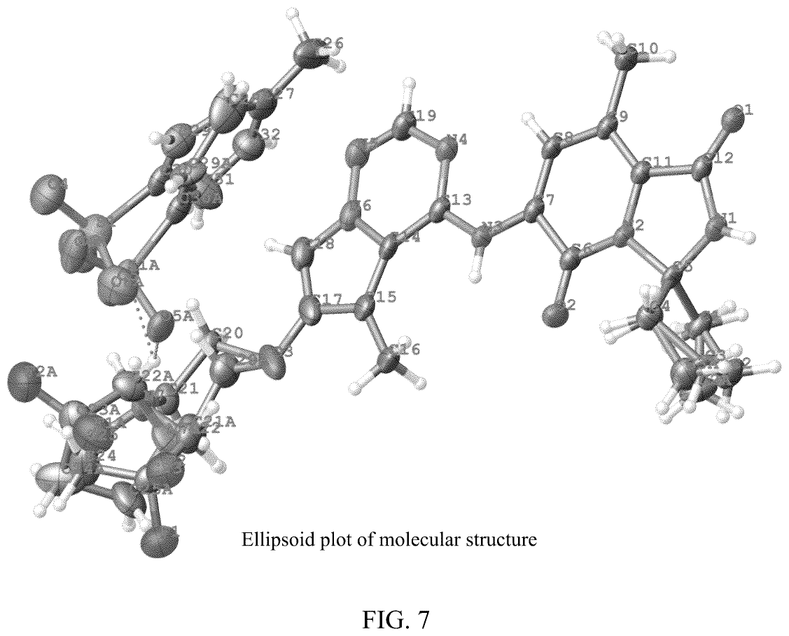

An appropriate amount of the compound of formula (II) was dissolved in 1 mL of dichloromethane/methanol (1:1) at room temperature, and the sample solution was placed in a 4 mL semi-sealed sample bottle to allow the solvents to be slowly volatilized at room temperature. The next day, a colorless massive crystal was obtained and tested by single-crystal X-ray diffraction. The analysis results of the single crystal structure are shown in FIG. 7.

### Example 3: Preparation of Crystal Form A of Compound of Formula (II)

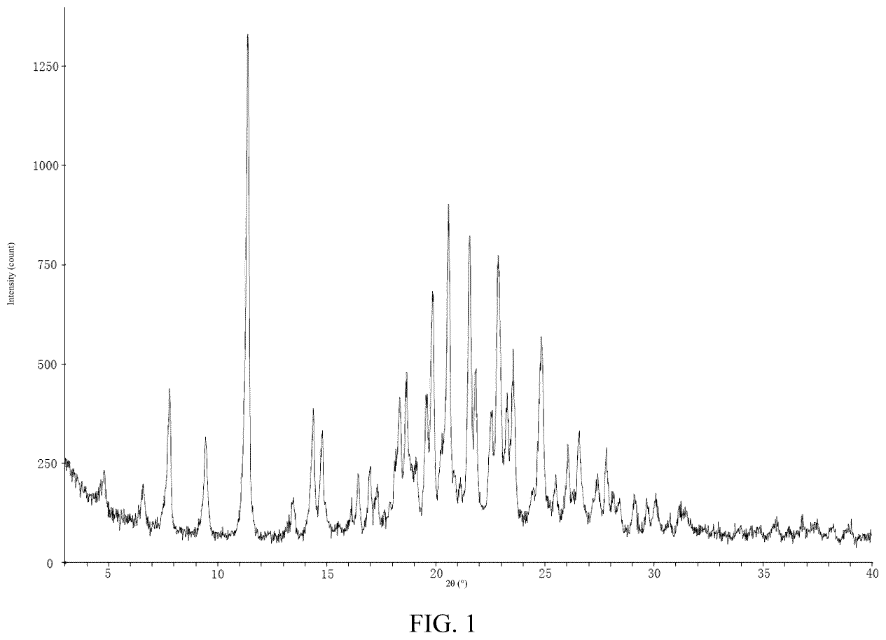

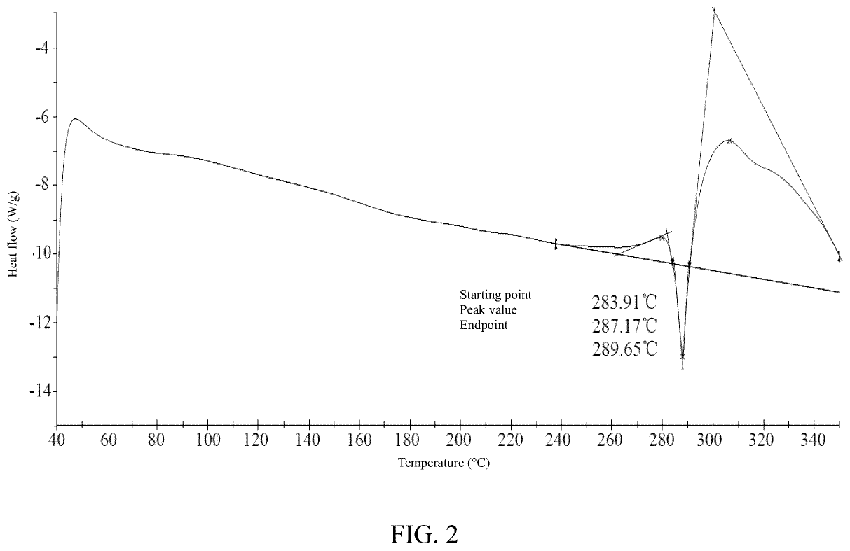

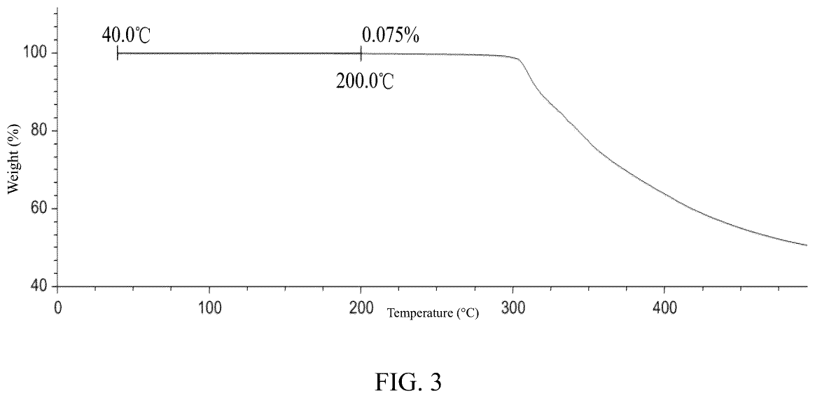

Acetonitrile (7.0 L), the compound of formula (I) (700.22 g), and p-toluenesulfonic acid monohydrate (272.25 g) were added successively to a reactor, with the internal temperature maintained at 20-30° C. The internal temperature of the reactor was raised to 45-55° C., and the reaction liquid was stirred for 4.5 h and filtered. The filter cake was rinsed with acetonitrile (0.7 L). The resulting filter cake was transferred into the reactor, acetonitrile (7.0 L) was added, and then the reaction liquid was stirred at 45-55° C. for 20 h and filtered. The filter cake was rinsed successively with acetonitrile (0.7 L) and tert-butyl methyl ether (1.4 L), and dried under vacuum and reduced pressure at a temperature not higher than 45° C. and a pressure ≤−0.1 MPa for 17 h to give crystal form A of the compound of formula (II). The XRPD, DSC, and TGA detection results are shown in FIG. 1, FIG. 2, and FIG. 3.

### Example 4: Crystal Form Screening Test of Compound of Formula (II) in Different Solvents

About 50 mg of the compound of formula (II) was weighed out separately and dissolved in different solvents, the mixtures were stirred at 50° C. for 3 h. The reaction liquids were cooled to room temperature, filtered for collection, and dried in vacuum, and the obtained solids were tested by XRPD. The test results are shown in Table 10 below:

TABLE 10
Stability test results of compound of formula (II) in different solvents

| | | | Crystal |
|---|---|---|---|
| No. | Solvent | Appearance | form |
| 1 | Methanol | Suspension | Crystal form A |
| 2 | Acetonitrile | Suspension | Crystal form A |
| 3 | tert-Butyl methyl | Suspension | Crystal form A |
| | ether | | |
| 4 | Ethanol | Suspension | Crystal form B |
| 5 | n-Heptane | Suspension | Crystal form B |

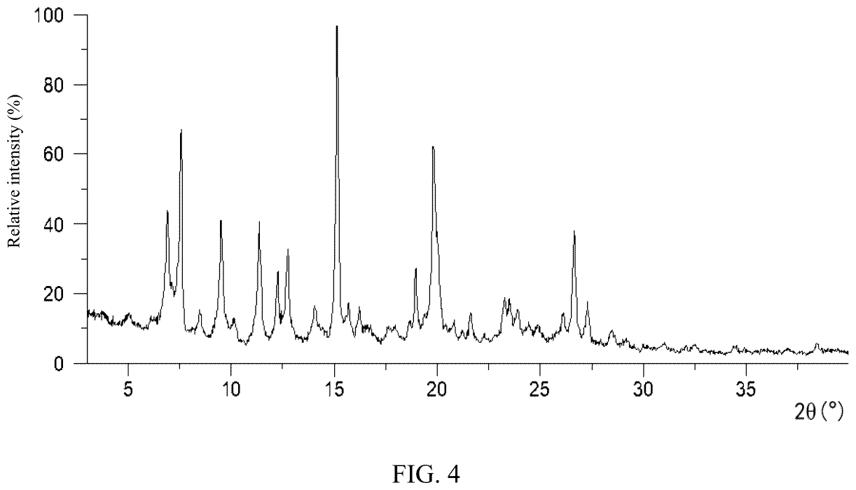

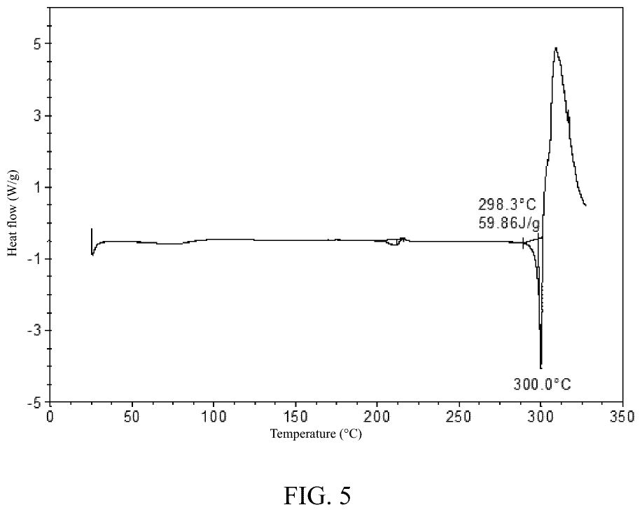

Conclusion: The compound of formula (II) forms crystal form A in methanol, acetonitrile, and tert-butyl methyl ether, and forms crystal form B in ethanol and n-heptane, wherein the XRPD and DSC detection results of the crystal form B are shown in FIG. 4 and FIG. 5 in sequence.

### Example 5: Study on Hygroscopicity of Crystal Form a of Compound of Formula (II)

### Materials

SMS DVS intrinsic dynamic vapor sorption instrument

### Procedures

The crystal form A of the compound of formula (II) (10-15 mg) was placed in a DVS sample tray for testing.

### Results

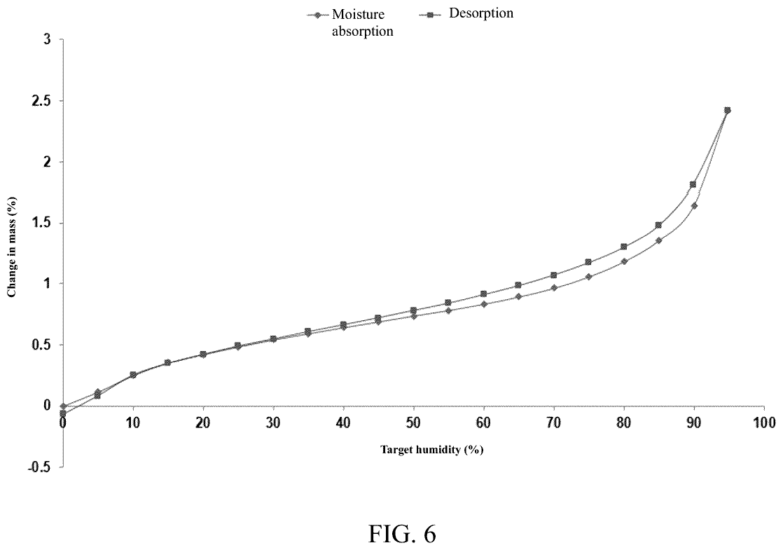

The DVS profile of the crystal form A of the compound of formula (II) is shown in FIG. 6, ΔW=1.179%.

### Conclusion

The crystal form A of the compound of formula (II) had a hygroscopic weight gain of 1.179% at 25° C. and 80% RH, had relatively weak hygroscopicity, and was not changed before and after the hygroscopic test, so that the crystal form A is a stable crystal form.

### Experimental Example 6: Study on Solid Stability Test of Crystal Form a of Compound of Present Disclosure

The crystal form A of the compound of the present disclosure was subjected to an accelerated lofting stability test and long-term lofting stability study concerning rules and requirements in “Guidelines for the Stability Test of APIs and Preparations” (General Principles 9001 of the Four Parts of the Chinese Pharmacopoeia, 2020 Edition), “Technical Guidelines for Stability Research of Chemical Drugs (APIs and Preparations) (Revised)” issued by the Center for Drug Evaluation of the National Medical Products Administration, and ICH Q1B.

### 1. Long-Term Accelerated Lofting Stability Test:

Each sample was separately put into a double-layer LDPE bag with each layer of LDPE bag sealed by a binding buckle. The bag was then put into an aluminum foil bag for heat sealing, and then put into a stability box under corresponding conditions for observation. The results of the solid stability test of the crystal form A of the compound are shown in Table 11.

TABLE 11
Results of solid stability test of crystal form A of compound of
present disclosure

| Test conditions | Time point | Crystal form |
|---|---|---|
| 25° C./60% RH | Day 0 | Crystal form A |
| | 12 months | Crystal form A |
| 40° C./75% RH | Day 0 | Crystal form A |
| | 6 months | Crystal form A |

Conclusion: the crystal form A of the compound of the present disclosure has good stability under the conditions of a long-term accelerated lofting stability test, and the crystal form A is a stable crystal form.

### Bioactivity Assay

### Assay Example 1: In Vitro Evaluation of Inhibitory Activity of Compound of Present Disclosure Against MNK2 Protein Kinase

Purpose of experiment: to test the inhibitory activity of the compound against MNK2 protein kinase Experimental materials: assay buffer: 8 mM 3-(N-morpholino)propanesulfonic acid, 0.2 mM disodium ethylenediaminetetraacetate, 0.01% polyoxyethylene lauryl ether, 5% glycerol, 0.1% β-mercaptoethanol and 1 mg of bovine serum albumin

Experimental operation: Mnk2 protein kinase inhibitory activity assays were performed using the KinaseProfiler™ service from Eurofins Pharma Discovery Services UK Limited. Serially diluted DMSO solutions containing the compound to be tested (3-fold serial dilution, starting from 10 μM), MNK2 (h) protein kinase and 0.33 mg/mL myelin basic protein were added to a freshly prepared buffer (pH=7.0), and then stirred homogeneously. The reaction was initiated by adding a mixture of ³³P-ATP (intensity of radioactivity: 10 μCi/μL) and 10 mM magnesium acetate. After the resulting mixture was reacted at room temperature for 40 min, the reaction was terminated by adding phosphoric acid to dilute to a concentration of 0.5%. 10 μL of the reaction liquid was filtered using a P30 filtermat, and then the filtermat was washed four times with 0.425% phosphoric acid for 4 min each, followed by washing once with methanol. After drying, the intensity of radioactivity was determined using the Filter-Binding method.

The protein kinase inhibitory activity of the compound was expressed as a percentage of the residual protein kinase activity relative to the blank substrate (DMSO alone). The Prism4 software package (GraphPad) was used to calculate the IC₅₀ value and curve. The specific information is shown in Table 12 below.

TABLE 12
Inhibitory activity (IC₅₀) of compound of present disclosure against
MNK2 protein kinase

| | IC₅₀ (nM) against |
|---|---|
| Compound No. | MNK2 |
| Compound of formula (I) | 17 |

Experimental conclusion: the compound of formula (I) of the present disclosure shows excellent inhibitory activity against MNK2 protein kinase.

### Assay Example 2: In Vitro Evaluation of Inhibitory Activity of Compound of Present Disclosure Against eIF4E Phosphorylation

Purpose of experiment: to test the inhibition (IC₅₀) of the compound against eIF4E phosphorylation of HCT116 cell strain.

Experimental materials: HCT116 cells (ATCC), RPM11640 medium (Life technology), fetal bovine serum (Hyclone), double antibodies (penicillin, streptomycin) (Millipore), phosphate buffer (Corning), 384-well cell plate (PerkinElmer), and AlphaLISA® SureFire® Ultra™ p-eIF4E (Ser209) Assay Kit (PerkinElmer).

Experimental operation: HCT116 cells were digested to make a cell suspension, which was then plated in a 96-well plate. The cell plate was then placed in an incubator for overnight incubation. The compound was diluted to a corresponding concentration and added to the cell plate, and the resulting mixture was incubated for 3 h. The cells were subsequently lysed with a lysis buffer, and the lysate was transferred to a 384-well plate. A mixed receptor was freshly prepared according to the kit instructions and added to the 384-well plate, and the mixture was incubated at room temperature for 1 h. Then a mixed donor was freshly prepared according to the kit instructions and added to the 384-well plate, and the resulting mixture was incubated at room temperature for 1 h. The signal was read on EnVision using the standard AlphaLISA program, a curve was fitted using Graphpad prism, and the IC₅₀ value was calculated. The specific information is shown in Table 13 below.

TABLE 13
Inhibitory activity (IC₅₀) of compound of present disclosure against
eIF4E phosphorylation

| | IC₅₀ (nM) against HCT116 cell strain |
|---|---|
| Compound No. | p-eIF4E |
| Compound of formula (I) | 8.6 |

Experimental conclusion: the compound of formula (I) of the present disclosure shows excellent inhibitory activity against eIF4E phosphorylation.

### Assay Example 3: In Vivo Efficacy Experiment of Compound of Present Disclosure for CT-26 Mouse Graft Tumor

Purpose of experiment: to determine in vivo efficacy of the compound for CT-26 mouse graft tumor

Experimental materials: CT-26 cells, RMPI-1640 medium containing 10% fetal bovine serum, and mice (female, Shanghai Sippe-Bk Lab Animal Co., Ltd.)

Experimental operation: CT-26 cells were cultured in RMPI-1640 medium containing 10% fetal bovine serum in an incubator at 37° C./5% CO₂. Tumor cells were subcultured, and after an appropriate concentration was reached and the tumor cells were in the logarithmic growth phase, the tumor cells were collected, counted and then resuspended in DPBS (phosphate buffer), and the concentration of the cell suspension was adjusted to 3×10⁶/mL for inoculation.

Establishment of mouse colon cancer graft tumor: cells were collected and adjusted to a concentration of 3×10⁶ cells/mL (resuspended in DPBS to obtain cell suspension), and 0.1 mL of tumor cells was injected subcutaneously at the right dorsal side of the mice under sterile conditions, and 3×10⁵ cells were inoculated for each mouse. After the tumor grew to a certain size, the length (a) and the width (b) of the tumor were measured by using a digital vernier caliper, and the tumor volume (TV) was calculated, wherein the calculation formula is as follows: TV=a×b²/2.

CT-26 tumor cell inoculation: on the day of inoculation, animals were grouped (8 animals for each) according to the body weight and subjected to drug administration separately, and the day of inoculation was considered as DO. When the tumor size reached about 60 mm³, the animals in the antibody groups were grouped according to the tumor size and the body weight. The body weight and tumor size of the animals were measured three times a week during the experiment, while clinical symptoms of the animals were observed and recorded daily, and for each administration, reference was made to the animal body weight measured most recently. The inhibitory effect of the compound on colon cancer graft tumor in mice was determined after 21-day administration at a dose of 30 mg/Kg QD (once daily), 90 mg/Kg QD (once daily) and 200 mg/Kg QD (once daily). The specific information is shown in Table 14 below.

The evaluation index of the anti-tumor activity is relative tumor proliferation rate T/C (%); if T/C (%)>40%, it suggests the drug is ineffective, and if T/C (%)≤40% and P<0.05 after statistical treatment, the drug is considered to be effective. The calculation formula of T/C (%) is as follows: T/C (%)=(T_{RTV}/C_{RTV})×100%. T_{RTV} is the relative tumor volume of the treatment group, and C_{RTV} is the relative tumor volume of the negative control group; TGI (%)^{a}=(1−average tumor volume at the end of administration of a treatment group/average tumor volume at the end of treatment of the solvent control group)×100%.

TABLE 14
In vivo anti-tumor efficacy of compound of present disclosure in
CT-26 graft tumor model

| Compound | Administration dose | TGI % | T/C % |
|---|---|---|---|
| Compound of formula (I) | 30 mg/Kg, QD | 63.57 | 36.43 |
| Compound of formula (I) | 90 mg/Kg, QD | 68.89 | 31.11 |
| Compound of formula (I) | 200 mg/Kg, QD | 68.51 | 33.31 |

Experimental conclusion: the compound of formula (I) of the present disclosure has a significant effect in inhibiting colon cancer graft tumor in mice.

1. A compound of formula (II),

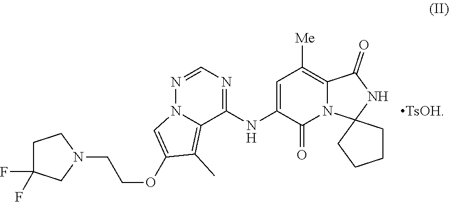

2. Crystal form A of a compound of formula (II), wherein an X-ray powder diffraction pattern of the crystal form A comprises characteristic diffraction peaks at the following 2θ angles: 7.78±0.20°, 11.38±0.20°, and 20.58±0.20°;

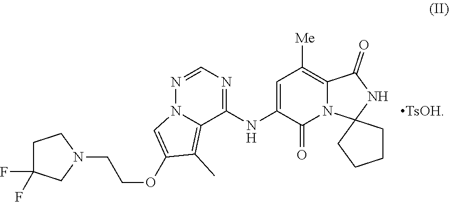

3. The crystal form A according to claim 2, wherein the X-ray powder diffraction pattern of the crystal form A comprises characteristic diffraction peaks at the following 2θ angles: 7.78±0.20°, 9.44±0.20°, 11.38±0.20°, 19.84±0.20°, 20.58±0.20°, 21.56±0.20°, 22.86±0.20°, and 24.82±0.20°.

4. The crystal form A according to claim 3, wherein the X-ray powder diffraction pattern of the crystal form A comprises characteristic diffraction peaks at the following 2θ angles: 6.58±0.20°, 7.78±0.20°, 9.44±0.20°, 11.38±0.20°, 14.38±0.20°, 18.66±0.20°, 19.84±0.20°, 20.58±0.20°, 21.56±0.20°, 22.86±0.20°, 23.54±0.20°, and 24.82±0.20°.

5. The crystal form A according to claim 4, wherein the X-ray powder diffraction pattern of the crystal form A comprises characteristic diffraction peaks at the following 2θ angles: 4.78°, 6.58°, 7.78°, 9.44° 11.38°, 13.48°, 14.38°, 14.80°, 16.42°, 17.00°, 17.32°, 18.34°, 18.66°, 19.08°, 19.60°, 19.84°, 20.28°, 20.58°, 21.56°, 21.84°, 22.52°, 22.86°, 23.26°, 23.54°, 24.46°, 24.82°, 25.50°, 26.04°, 26.58°, 27.42°, 27.82°, 28.07°, 28.42°, 29.08°, 29.66°, 30.08°, 31.20°, 31.42°, 38.22°, and 39.04°.

6. The crystal form A according to claim 5, wherein the XRPD pattern of the crystal form A is substantially as shown in FIG. 1.

7. The crystal form A according to claim 2, wherein a differential scanning calorimetry curve of the crystal form A has an endothermic peak at 287.17±3° C.

8. The crystal form A according to claim 7, wherein a DSC profile of the crystal form A is shown in FIG. 2.

9. The crystal form A according to claim 2, wherein a thermogravimetric analysis curve of the crystal form A shows a weight loss of 0.075% at 200.0±3° C.

10. The crystal form A according to claim 9, wherein a TGA profile of the crystal form A is shown in FIG. 3.

11. A preparation method for crystal form A of the compound of formula (II) according to claim 2, comprising the following steps:
(a) adding the compound of formula (II) to a solvent to form a suspension;
(b) stirring the suspension at 40-55° C. for 2-25 h; and
(c) filtering the suspension and then drying the filter cake in vacuum at 30-45° C. for 10-24 h; wherein the solvent is selected from methanol, acetonitrile, and tert-butyl methyl ether.

12. Crystal form B of a compound of formula (II), wherein an X-ray powder diffraction pattern of the crystal form B comprises characteristic diffraction peaks at the following 2θ angles: 7.55±0.20°, 15.13±0.20°, and 19.82±0.20°;

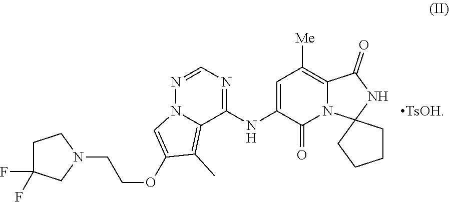

13. The crystal form B according to claim 12, wherein the X-ray powder diffraction pattern of the crystal form B comprises characteristic diffraction peaks at the following 2θ angles: 6.89±0.20°, 7.55±0.20°, 9.50±0.20°, 11.35±0.20°, 12.72±0.20°, 15.13±0.20°, 19.82±0.20°, and 26.63±0.20°.

14. The crystal form B according to claim 13, wherein the X-ray powder diffraction pattern of the crystal form B comprises characteristic diffraction peaks at the following 2θ angles: 6.89±0.20°, 7.55±0.20°, 9.50±0.20°, 11.35±0.20°, 12.24±0.20°, 12.72±0.20°, 15.13±0.20°, 18.94±0.20°, 19.82±0.20°, 23.25±0.20°, 26.63±0.20°, and 27.27±0.20°.

15. The crystal form B according to claim 14, wherein the X-ray powder diffraction pattern of the crystal form B comprises characteristic diffraction peaks at the following 2θ angles: 4.98°, 6.89°, 7.55°, 8.46°, 9.50°, 10.12°, 11.35°, 12.24°, 12.72°, 14.05°, 15.13°, 15.65°, 16.20°, 17.79°, 18.94°, 19.82°, 20.76°, 21.61°, 23.25°, 23.87°, 26.09°, 26.63°, 27.27°, 28.45°, 29.12°, 30.95°, 32.32°, 34.62°, and 38.41°.

16. The crystal form B according to claim 15, wherein the XRPD pattern of the crystal form B is substantially as shown in FIG. 4.

17. The crystal form B according to claim 12, wherein a differential scanning calorimetry curve of the crystal form B has an endothermic peak at 300.0±3° C.

18. The crystal form B according to claim 17, wherein a DSC profile of the crystal form B is shown in FIG. 5.

19. A method for inhibiting MNK1/2, which comprises administering to a subject a therapeutically effective amount of the crystal form A according to claim 2.

20. A method for treating colorectal cancer, which comprises administering to a subject a therapeutically effective amount of the crystal form A according to claim 2.
